(19) United States
(12) Patent Application Publication
(10) Pub. No.: US 2018/0263497 A1
(43) Pub. Date: Sep. 20, 2018

(54) PROTECTION OF INDUCTIVE COMMUNICATION SYSTEM FROM AN MRI GRADIENT FIELD USING AN INDUCTORS CORE SATURATION CHARACTERISTICS

(71) Applicant: BIOTRONIK SE & CO. KG, BERLIN (DE)

(72) Inventors: JAMES W. HESS, ALOHA, OR (US); HABIB HOMAYOUN, BEAVERTON, OR (US); JEFFREY A. VON ARX, LAKE OSWEGO, OR (US)

(21) Appl. No.: 15/903,701

(22) Filed: Feb. 23, 2018

Related U.S. Application Data
(60) Provisional application No. 62/471,384, filed on Mar. 15, 2017.

(51) Int. Cl. A61B 5/00

(52) U.S. Cl. CPC A61B 5/0031; A61B 5/686; A61B 5/0033

(57) ABSTRACT

An implantable medical device includes a coil which is configured for transmission and/or reception of RF fields for communication with external devices. The coil has a conductor that is wound around a core. The core is adapted to saturate in the presence of a static magnetic field or a B0-field generated by an MRI machine thereby reducing the voltage induced in the coil by an RF field and/or by a gradient field generated by the MRI machine.

### CROSS-REFERENCE TO RELATED APPLICATION

This application claims the benefit, under 35 U.S.C. § 119(e), of provisional patent application No. 62/471,384 filed Mar. 15, 2017; the prior application is herewith incorporated by reference in its entirety.

### TECHNICAL FIELD

The invention relates to an implantable medical device, i.e., a medical device that can be implanted into a human or animal body.

### BACKGROUND

Such devices frequently use a magnetic field for communication between a programmer and the implantable/implanted medical device. On the device side, an inductor such as a coil, particularly an air core coil, can be used to both receive and transmit information.

U.S. Pat. No. 8,253,555 B2 discloses a pacemaker with an RFID system for implant identification. Furthermore, U.S. Pat. No. 9,037,258 B2 describes MRI protection systems for implants.

Particularly, when an medical device of the afore-mentioned kind is placed in a magnetic resonance imaging (MRI) device, e.g. when a person carrying the medical device/implant is examined by means of the MRI machine, the AC fields (gradient field) induce a voltage on the air coil mentioned above. This voltage can be sufficient to cause damage to the device. Therefore, clamping diodes have been used in the prior art to divert the current and clamp the voltage induced to a safe level.

Disadvantageously, such diodes require an additional installation space which is not desirable when aiming at miniaturization of the medical device.

Alternatively, the integrated circuits (ICs) of the implantable medical device can be designed to withstand the induced voltage and/or currents. But this requires excessively large circuitry on the respective IC capable of handling the voltages and/or current, which ultimately is not aligned with the miniaturization needs that have to be met with implantable medical devices.

### SUMMARY OF THE INVENTION

Based on the above, the problem to be solved by the present invention is to provide an implantable medical device that offers protection with respect to said fields that are generated by MRI machines but only requires a relatively small installation space for its components.

This and other objects of the invention are achieved by an implantable medical device as claimed.

There is provided, in accordance with the invention, an implantable medical device, comprising:

a coil which is configured for transmission and/or reception of RF fields for communication with external devices, that is, for transmission and/or reception of magnetic fields for communication with external devices;

the coil comprises a conductor that is wound around a core, wherein the core is adapted to saturate in the presence of a static magnetic field or a B0-field generated by an MRI machine, thereby reducing the voltage induced on the coil by an RF field and/or by a gradient field generated by the MRI machine.

Preferably, the coil comprises a conductor that is wound around a core, wherein the core comprises a ferromagnetic material.

In an embodiment of the present invention, the coil is configured for transmission and/or reception of radiofrequency (RF) fields for communication with external devices.

Particularly, in case this communication coil is placed in an MRI machine, the large static field (e.g. B0 field) of the MRI machine will saturate the communication coil's core, and thereby reduce the voltage and current induced into the coil to a very small level. As a consequence, the clamping diodes used in the prior art are no longer needed to protect the medical device and can therefore be omitted. Particularly, the medical implant device according to the invention does not comprise clamping diodes connected in parallel to the coil for protection of the coil. In embodiments where the high voltages generated in the environment of the MRI machine are handled via an integrated circuit (IC) for protecting the medical device, application of the present invention can save protection circuitry for said IC.

Particularly, magnetic resonance imaging (MRI) is a medical imaging technique used in radiology to form pictures of the anatomy and the physiological processes of the body in both health and disease. Typically, MRI machines (scanners) use strong magnetic fields, radio waves, and field gradients to generate images of the inside of the body.

The B0-field is the static magnetic field of an MRI machine that is used to polarize spins and create magnetization. In contrast thereto, the B1-field generated by the MRI machine is an RF energy field that is applied perpendicular to the longitudinal axis (B0-field) in order to perturb the magnetization, e.g. to create excitation pulses etc. Moreover, magnetic field gradients are applied which causes the static field to vary across scanned volume so that different spatial locations become associated with different procession frequencies, allowing spatial encoding and isolation of specific portions of the body for MR imaging.

Particularly, according to an embodiment of the present invention, the core is adapted to saturate in the presence of a static magnetic field, particularly a B0-field generated by an MRI machine, which MRI machine is configured for performing MRI on a human or animal patient into which patient said implantable medical device is implanted, thereby reducing the voltage induced on the coil by RF and/or gradient fields generated by the MRI machine. In the preferred embodiment, the core is adapted to saturate at a field strength much less then the B0-field (e.g. Bsaturation<<0.1*B0). This is to ensure that core saturation occurs even during MRI scans in which the implant is outside of the MRI bore (e.g. as likely occurs during a foot scan when the implant is in the pectoral region of the chest).

Particularly, according to an embodiment, the coil is a bobbin coil. Typically, coils wound on a core with a shape like a bobbin are called bobbin coils. The bobbin shaped core has a thin middle which the coil is wound on, and two end caps. According to other embodiments of the invention, the core of the coil may have other shapes, as e.g. a rod core. The core may have a cylindrical or a cuboid shape.

Further, according to an embodiment of the implantable medical device according the invention, the implantable medical implant device comprises a communication circuit configured to communicate with an external device, particularly a near field communication circuit (e.g. 32 kHz, and/or . . . ), which comprises said coil adapted for protection against RF and gradient fields generates by an MRI machine (see above).

Further, according to an embodiment of the implantable medical device according the invention, said ferromagnetic material is ferrite.

Further, according to an embodiment of the implantable medical device according the invention, the ferromagnetic material is a μ-metal (also denoted mu-metal), e.g. a nickel-iron soft magnetic alloy with very high permeability. Preferably, a material should be chosen with higher permeability than ferrite. In a particular embodiment, a material with chemical composition of with 80% Nickel, 4% Molybdenum, 15% Iron, and 1% trace material is chosen as core material. In a particular embodiment, a material with chemical composition of 80-81% Nickel, 4.5-6% Molybdenum, 0.05-0.4% Silicon, 0-0.5% Manganese, 0.01% Carbon, Iron balance is chosen as core material.

Further, according to an embodiment of the implantable medical device according the invention, the core or said ferromagnetic material has a relative permeability greater than 250, particularly greater than 1000, particularly greater than 5000, particularly greater than 10000, particularly greater than 15000.

Further, according to an embodiment of the implantable medical device according the invention, the core or said ferromagnetic material has a relative permeability that is smaller than 30000, particularly smaller than or equal to 20000.

Further, according to an embodiment of the implantable medical device according the invention, the core or said ferromagnetic material is adapted such that is saturates in a magnetic field that is smaller than 7 T, particularly smaller than 3 T, particularly smaller than 1.5 T, particularly smaller than 150 mT, particularly smaller than 50 mT.

Further, according to an embodiment of the implantable medical device according the invention, the core or said ferromagnetic material is adapted such that it does not saturate in a magnetic field that is smaller than 100 mT, particularly smaller than 30 mT, particularly smaller than 10 mT, particularly smaller than 1 mT.

Further, according to an embodiment of the implantable medical device according the invention, the diameter of the coil is smaller than 3.5 mm, and/or the length of the coil in an axial direction of the coil is smaller than 1.75 mm. In a preferred embodiment of the invention, the coil has a square footprint with the dimensions 3 mm×3 mm or smaller, wherein the length of the coil in direction of the coil axis is 1.5 mm or smaller. In an embodiment, the coil has a circular footprint with a diameter of 3 mm.

Further, according to an embodiment of the implantable medical device according the invention, the core comprises a diameter that is smaller than or equal to 1 mm. The diameter of the core is understood as the diameter which runs perpendicular to the coil axis when the core is placed within the coil.

Other features which are considered as characteristic for the invention are set forth in the appended claims.

Although the invention is illustrated and described herein as embodied in an implantable medical device with a protection of the inductive communication system from an MRI gradient field using an inductor's core saturation characteristics, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWING

FIG. 1 shows a representation of the circuit used in the prior art, which comprises an air core inductor and two clamping diodes for protecting the circuit e.g. against RF/gradient fields;

FIG. 2 shows a schematic representation of an implantable medical device according to the present invention, where the clamping diodes are omitted and the inductor is formed by a coil having a core out of a ferromagnetic material;

FIG. 3 illustrates the difficulty when using an air core coils as communication coil in an implantable medical device;

FIG. 4 schematically shows the use of a coil having a core according to the present invention;

FIG. 5 schematically shows the effect of an MRI B0-field on the coil of an implantable medical device according to the present invention; and

FIG. 6 shows a further embodiment/block diagram of an implantable medical device according to the present invention.

### DETAILED DESCRIPTION

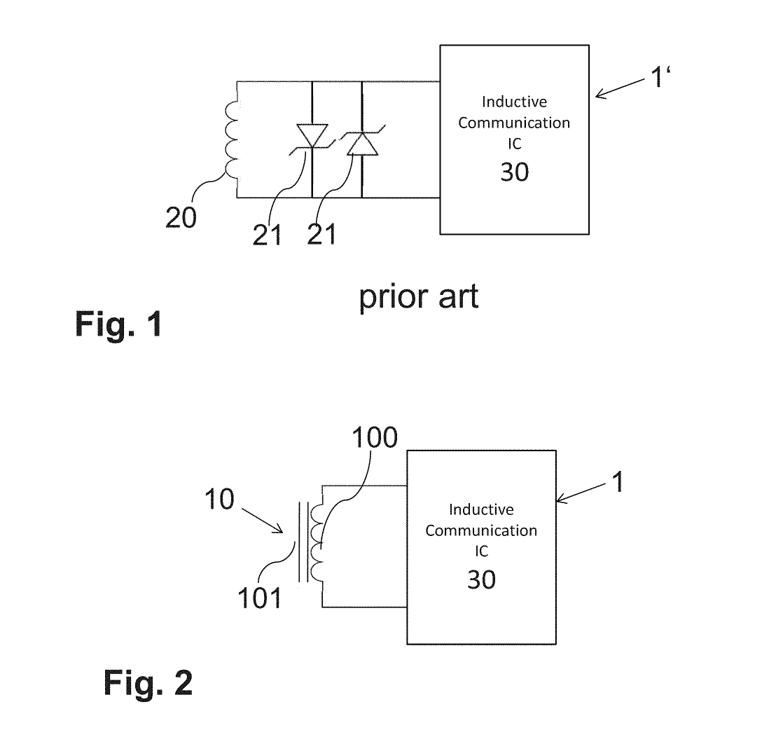

Referring now to the figures of the drawing in detail, FIG. 1 shows a schematic representation of a prior art implantable medical device 1′ having a communication coil 20, wherein the device is protected against RF fields, particularly gradient fields of an MRI machine as described above by means of two clamping diodes 21 which are connected to the coil 20 and the remaining communication circuit 30 as indicated in FIG. 1.

In contrast thereto, FIG. 2 shows the use of a communication coil 10 having a core 101 around which a conductor 100 of the coil 10 is wound, wherein the core 10 comprises a ferromagnetic material for protection against said MRI fields.

Particularly, in order to specifically achieve a coil 10 that comprises good communication performance with built-in protection from MRI induced currents and voltages, the core 101 is carefully chosen, particularly such that it comprises a high permeability with a low saturation point. The core material can be chosen to have a magnetic saturation at magnetic field strengths which are comparatively far below the field strength of a typical MRI static magnetic field B0. For example, the core material can be chosen to saturate at 150 mT or approximately at 150 mT. In an embodiment, the material saturates at a magnetic field strength from 150 mT-250 mT or at a magnetic field strength from 30 mT to 100 mT. According to an embodiment of the present invention, the core material is chosen such that it is fully magnetically saturated when it is brought in proximity of 50 cm, 40 cm, 30 cm, 20 cm, or 10 cm to the isocenter of the MRI bore or closer. In an embodiment of the present invention, the core material is chosen such that it is fully magnetically saturated when it is brought in proximity of between 130 cm 190 cm from the isocenter of the MRI bore. In the context of the present invention, the isocenter (or magnetic isocenter) of an MRI machine is understood as the center point of the magnetic field of the MRI machine. In case the MRI bore is cylindrical, the isocentrer is at the center of the bore.

Particularly, mu-metal can be used as material for the core 101, since it has an extremely high permeability and is relatively easy to saturate. Particularly, it is beneficial to choose a core 101 whose saturation point is not too low, because the induced magnetic field in the coil 10 during communications shall particularly not saturate the core 101. Otherwise, the communication can be lost. Particularly, also the self-inductance of the coil 10 is chosen such that self-saturation is avoided. Furthermore, some implantable medical devices have the capability to connect wirelessly with a programmer device via a magnetic switch. The magnetic switch is activated when a activation means having a magnet (e.g. a magnetic wand) is brought in close proximity to the implantable device. The core material according to the present invention should be chosen such that it is not saturated by the magnetic field of said activation means.

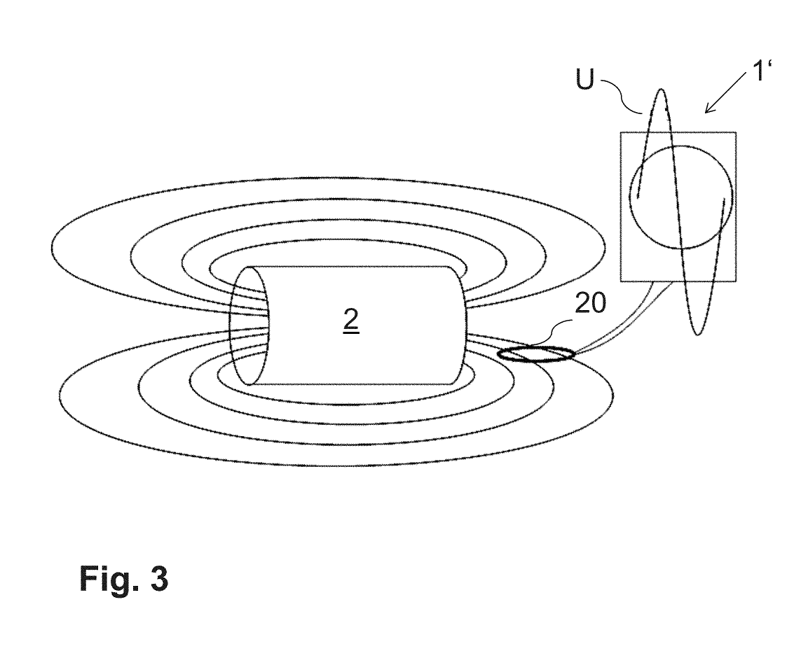

The air core inductors 20 used in the prior art as shown in FIG. 1 are designed to have a large “core” area, so as to maximize the flux lines from the wand through the inductor. This also maximizes an MRI machines's RF field and gradient field flux lines through the inductor, thus maximizing the voltage U generated when the air core coil 20 is placed in an MRI machine 2, as shown in FIG. 3.

In contrast thereto, the present invention uses an inductor 10 with a metal core. While air has a relative permeability of 1, metals have relative permeabilities that can be as high as 1,000,000. Using the metal core 101 allows the physical size of the inductor 10 to be reduced dramatically. By using particularly a mu-metal with a relative permeability in the range of 20,000 one has a physically much smaller device.

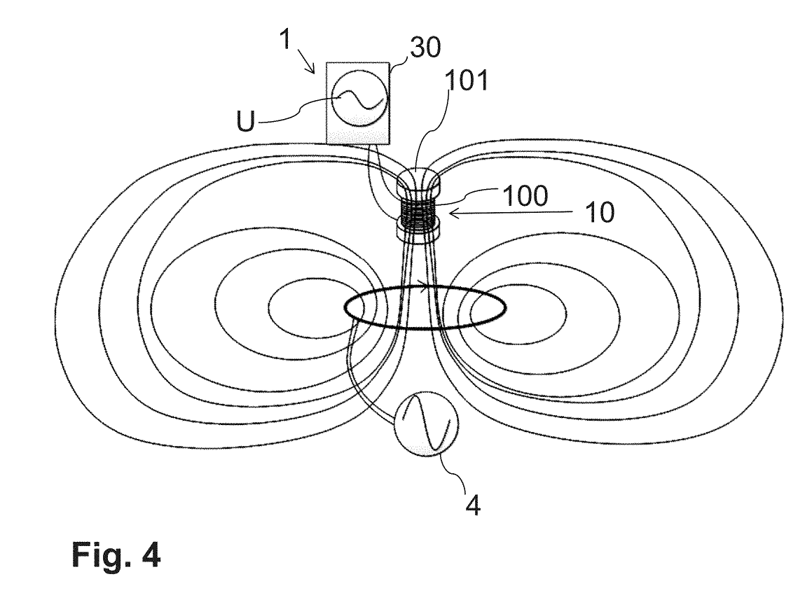

When the core material is not saturated, as shown in FIG. 4, during normal operation, the inductance is much higher, and the core 101 of the coil 10 of the communication circuit 30 (here e.g. receiver) has a tendency to pull flux into it, thus increasing the voltages U generated by the coil 10 during communication with a transmitter 4.

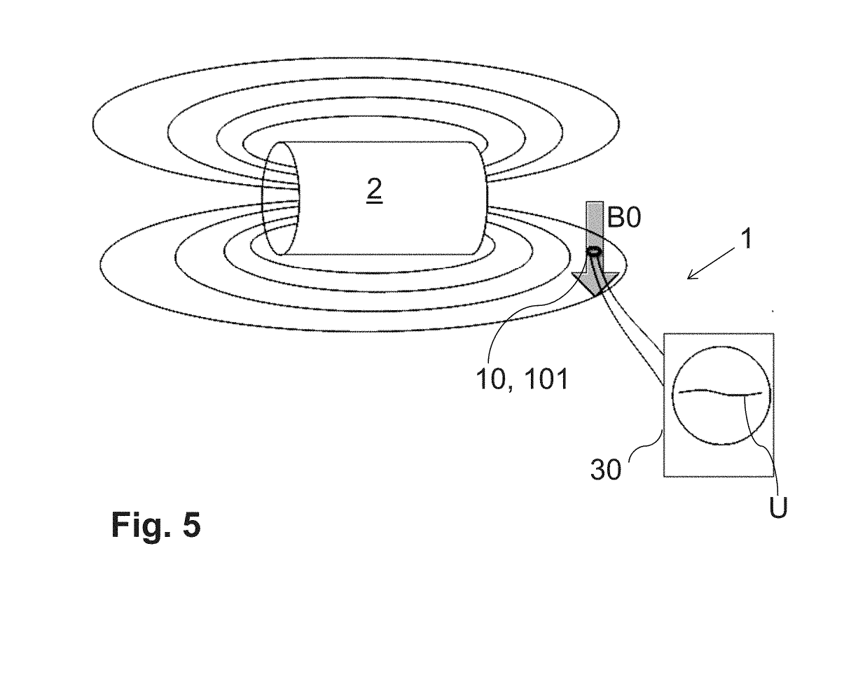

When the device 1 is placed in an MRI machine 2 as shown in FIG. 5, the static field (e.g. B0-field) causes the core's material to saturate. When saturated, the material behaves as if it was air with a relative permeability of 1 rather than the actual value of the core (e.g. 20,000). Then, the core 101 no longer pulls magnetic flux into it. Coupled with its much smaller coil size, the total flux from the MRI machines RF field and gradient field is much smaller. Thus the voltages induced on the coil are much smaller and need simpler or even no protection mechanisms at all. In turn, this simplifies the overall system design and reduces cost.

Particularly, in an example, the size of the coil 10 is 3 mm by 3 mm by 1.5 mm, and the core is formed out of a mu-metal so that the core 101 has a relative permeability of 20,000, wherein the core diameter is 1 mm, the inductance (non saturated) is 70 mH and the resistance is 1 kΩ.

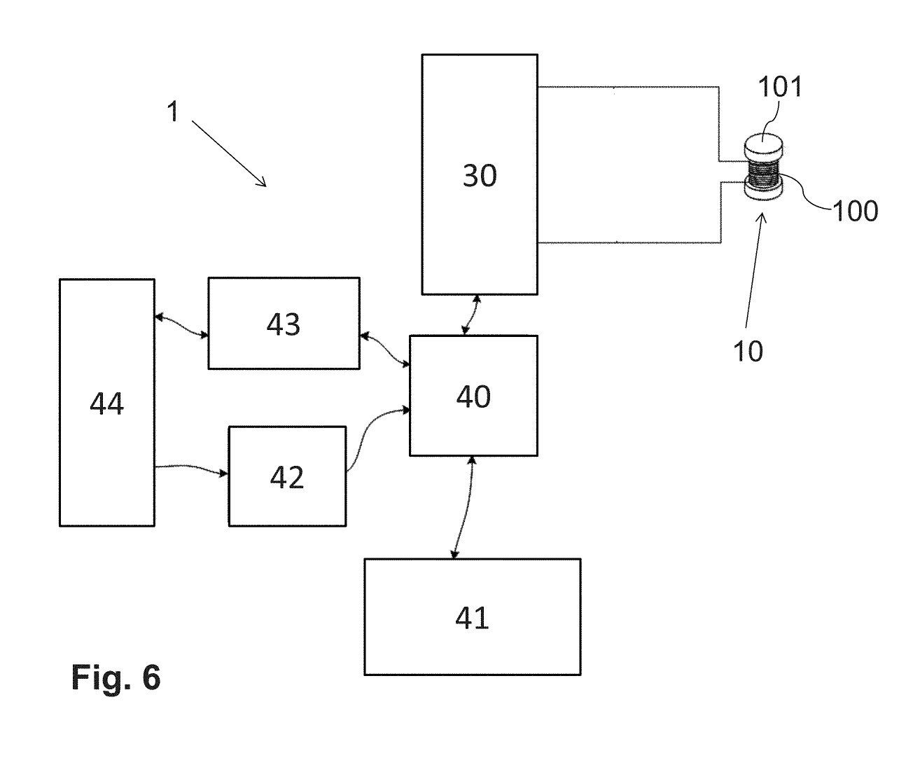

Finally, FIG. 6 shows a further embodiment of the implantable medical device according to the present invention, wherein the device 1 comprises a coil 10 which is configured for transmission and/or reception of magnetic fields or RF fields for communication with external devices, wherein the coil 10 comprises a conductor 100 that is wound around a core 101, wherein the core 101 comprises a ferromagnetic material, particularly a mu-metal or ferrite, particularly as specified herein.

The coil 10 forms part of a magnetic communication circuit 30, which is connected to a control unit 40. The magnetic communication circuit 30 consists of a transmit circuit and a receive circuit for transmitting and receiving signals for communication. The communication signals are preferably based on changing magnetic fields or, alternatively on RF fields. The transmit circuit is configured to drive the coil 10 with an alternating voltage or current at the communication frequency during periods of transmission. The transmit circuit is also configured to modulate the driving signal during periods of transmission to encode bits. The receive circuit is configured to amplify signals induced on the coil during periods of receiving. It is also configured to demodulate the received signals to recover the transmitted signal. The control unit 40 in turn is connected to a memory 41, a sensor unit 42, and an RF transceiver 43. Both, the RF transceiver 43 and the sensor unit 42 are connected to a front end circuit 44.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

1. An implantable medical device, comprising:
a coil configured for transmission and/or reception of RF fields for communication with external devices;
said coil having a core and a conductor wound around said core;
said core being configured to saturate upon being subjected to a static magnetic field or a B0-field generated by an MRI machine and to thereby reduce a voltage induced in said coil by an RF field and/or by a gradient field generated by the MRI machine.

2. The implantable medical device according to claim 1, wherein said core comprises a ferromagnetic material.

3. The implantable medical device according to claim 1, further comprising a communication circuit including said coil configured to communicate with an external device.

4. The implantable medical device according to claim 3, wherein said communication circuit is a near field communication circuit.

5. The implantable medical device according to claim 2, wherein said ferromagnetic material is a ferrite.

6. The implantable medical device according to claim 2, wherein said ferromagnetic material is a μ-metal.

7. The implantable medical device according to claim 2, wherein said ferromagnetic material of said core has a relative permeability greater than 250.

8. The implantable medical device according to claim 7, wherein said ferromagnetic material of said core has a relative permeability greater than 1000.

9. The implantable medical device according to claim 7, wherein said ferromagnetic material of said core has a relative permeability greater than 5,000 or a relative permeability greater than 10,000.

10. The implantable medical device according to claim 7, wherein said ferromagnetic material of said core has a relative permeability greater than 15,000.

11. The implantable medical device according to claim 2, wherein said ferromagnetic material of said core has a relative permeability that is smaller than 30,000.

12. The implantable medical device according to claim 11, wherein said ferromagnetic material of said core has a relative permeability that is smaller than or equal to 20,000.

13. The implantable medical device according to claim 2, wherein said core or said ferromagnetic material of said core is configured to saturate in a magnetic field that is smaller than 7 T.

14. The implantable medical device according to claim 14, wherein said core or said ferromagnetic material of said core is configured to saturate in a magnetic field that is smaller than 3 T or in a magnetic field that is smaller than 1.5 T or in a magnetic field that is smaller than 150 mT.

15. The implantable medical device according to claim 14, wherein said core or said ferromagnetic material of said core is configured to saturate in a magnetic field that is smaller than 50 T.

16. The implantable medical device according to claim 2, wherein said core or said ferromagnetic material of said core is configured not to saturate in a magnetic field that is smaller than 100 mT.

17. The implantable medical device according to claim 16, wherein said core or said ferromagnetic material of said core is configured not to saturate in a magnetic field that is smaller than 30 mT or in a magnetic field that is smaller than 10 mT.

18. The implantable medical device according to claim 16, wherein said core or said ferromagnetic material of said core is configured not to saturate in a magnetic field that is smaller than 1 mT.

19. The implantable medical device according to claim 1, wherein a diameter of said coil is smaller than 3.5 mm and/or wherein a length of said coil in an axial direction of said coil is smaller than 1.75 mm.

20. The implantable medical device according to claim 1, wherein said core has a diameter that is smaller than or equal to 1 mm.
